SEQUENCE LISTING (2 sequences)

SEQ ID NO: 1 - protein, 728 aa, Human
MWVTKLLPALLLQHVLLHLLLLPIAIPYAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIVNLDHPVISCAKTKQLRVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

SEQ ID NO: 2 - protein, 720 aa, Human
MWVTKLLPALLLQHVLLHLLLLPIAIPYAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIVNLDHPVISCAKTKQLRVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKV

(19) United States
(12) Patent Application Publication
(10) Pub. No.: US 2010/0279934 A1
(43) Pub. Date: Nov. 4, 2010

(54) TOPICAL COMPOSITIONS FOR DELIVERY OF PROTEINS AND PEPTIDES

(75) Inventor: Ake Lindahl, Malmo (SE)

Correspondence Address: KNOBBE MARTENS OLSON & BEAR LLP, 2040 MAIN STREET, FOURTEENTH FLOOR, IRVINE, CA, 92614, US

(73) Assignee: KRINGLE PHARMA, INC., Toyonaka, Osaka (JP)

(21) Appl. No.: 12/810,470

(22) PCT Filed: Dec. 19, 2008
(86) PCT No.: PCT/IB08/03954

§ 371 Date: Jun. 24, 2010

Related U.S. Application Data
(60) Provisional application No. 61/018,640, filed on Jan. 2, 2008.

(51) Int. Cl. A61K 38/18; A61P 17/00; A61P 17/02

(52) U.S. Cl. 514/9.5

(57) ABSTRACT

Aspects of this invention relate to compositions and methods for the stabilization, storage, and delivery of biologically active agents, in particular peptides and nucleic acids. Particularly preferred embodiments include compositions that comprise a recombinant naturally occurring human hepatocyte growth factor (HGF), such as dHGF, a five amino acid truncated HGF variant, and at least one monoglyceride that remains in crystalline form at body temperature. Optionally, a gelling agent such as, hydroxyethylcellulose, and/or an antipathogenic compound (e.g., bupivacaine) can be included in the composition. Methods of use of these compositions to improve, ameliorate, or treat skin conditions are also embodiments.

### FIELD OF THE INVENTION

Aspects of this invention relate to compositions and methods for the stabilization, storage, and delivery of proteins and peptides, in particular, hepatocyte growth factor (HGF) and variants thereof, and methods of use of such stabilized compositions to inhibit, ameliorate, or treat human ailments, such as skin ulcers and skin cancer or pre-cancer conditions.

### BACKGROUND OF THE INVENTION

Protein/peptide-based drugs present unique challenges for drug delivery. The susceptibility of proteins/peptides to denaturation in the gastrointestinal tract makes oral administration unfavourable. Consequently, protein/peptide-based drugs are generally administered systemically in the form of sterile injectable solutions. Since proteins/peptides have very short pharmacokinetic half-lives in the blood stream, being quickly metabolized and cleared, parenteral administration is also inefficient. Accordingly, many investigators are seeking to develop alternative approaches to stabilize, store, and deliver protein/peptide-based therapeutics.

In conventional wound treatments, e.g., after surgery or to treat skin ulcers, sterile protein/peptide containing solutions are infused into the wound site. In these applications, the low viscosity of the solution complicates administration and the dissolved protein/peptide is easily degraded, especially during storage. The manufacture of protein/peptide-based therapeutics, wherein the active ingredient is present in the form of a solid powder that upon exposure to a liquid will form a semisolid formulation has been described (see e.g., U.S. Pat. No. 5,192,743, herein expressly incorporated by reference in its entirety). Current products in reconstitutable form that contain sensitive proteins/peptides generally have poor stability, however, particularly if the product is stored at room temperature.

Another difficulty faced by investigators in this field concerns the application of these products to open wounds in a clinical setting. That is, many open-wounds including skin ulcers, abrasions, cosmetic blemishes, or lacerations, contain a variety of pathogens (e.g., bacteria, fungi, or other microbes or microorganisms) some of which are resistant to conventional treatment approaches (e.g., antibiotic therapy). The species of pathogen is often of “patient origin” and will not create a systemic infection but the presence of the pathogen is sufficient to prevent the healing of the wound. Some of these pathogens produce proteases that degrade the proteins/peptides present in the therapeutic, for example.

The poor stability of the protein/peptide-based products can be related to the presence of lipids and surfactants in the formulation, which interact with the proteins or peptides thereby altering the structure of the molecule and reducing or inhibiting its function. Small amounts of lipids and surfactants may change the three dimensional structure of a protein or peptide and thereby reduce the efficacy of the product. The presence of monoglycerides in infant formula emulsions has been shown to reduce the heat stability of the product, for example (see e.g., McSweeney et al., Food Hydrocolloids, Volume 22, Issue 5, July 2008, Pages 888-898).

The poor stability of protein/peptide-based products also makes it very difficult to accurately provide a therapeutic dose of the product. That is, because the rate of degradation or destabilization of the protein or peptide (the active ingredient) may differ from patient to patient, it is difficult to formulate safe and effective protein/peptide-based products. Oftentimes, these problems are accounted for by providing more of the active ingredient (overage) but this approach can be detrimental especially in formulations, wherein the pharmacological activity of the product at an increased concentration of the active ingredient is different than that exhibited at a low concentration.

Such is the case with current hepatocyte growth factor (HGF)-containing products. HGF is known to accelerate the growth of normal epitheliocytes and to improve cell motility (see e.g., U.S. Pat. No. 5,342,831; issued Aug. 30, 1994). Although HGF-containing wound healing products have been contemplated for almost 20 years, the development of these products has been hindered by the poor stability of the molecule and its unique biological properties. HGF is a relatively unstable molecule having a half-life of only about 10 minutes in the blood (see e.g., U.S. Pat. No. 7,247,620; issued Jul. 24, 2007). HGF's stimulatory activity appears to be concentration dependent, wherein maximum cell proliferation was seen at 2.5 and 5 ng/ml and higher concentrations were reported to be uneffective (see e.g., Bussolino et al., J. of Cell Biol. Vol. 119, No. 3, 629-641 (1992)). These factors have led some in the field to conclude that a specific and effective method for administration of HGF protein, effective dosing and the like has yet to be found (see e.g., U.S. Pat. No. 7,247,620; issued Jul. 24, 2007). Accordingly, the development of new formulations of stabilized protein/peptide-based therapeutics, especially stabilized formulations containing HGF, is manifest.

### SUMMARY OF THE INVENTION

It has been discovered that the protein/peptides (“active ingredients”) in protein/peptide-based therapeutics, especially HGF-containing formulations, can be stabilized by including an effective amount of a crystalline monoglyceride (e.g., an α or β-crystalline monoglyceride), which may have a carbon chain length of 10, 11, 12, 13, 14, 15, 16, 17, or 18 carbons, desirably a carbon chain length between 10-16 carbons (e.g., 12, 13, 14, 15, or 16 carbons), preferably a carbon chain length that is 12, 13, or 14 carbons, and most preferably, 12 or 14 carbons. Accordingly, aspects of the invention described herein concern a composition that comprises a protein or a peptide (e.g., an HGF molecule, such as full-length HGF or dHGF (a naturally occurring five amino acid truncated form of HGF) or other naturally occurring HGF molecules or variants thereof (e.g., NK1, dNK1, NK2, dNK2, NK3, dNK3, NK4 or dNK4)) and one or a mixture of a plurality of monoglycerides (e.g., an α and/or β-crystalline monoglycerides) that remain in a crystalline form at temperatures greater than or equal to 15° C., desirably greater than or equal to 20° C., and, preferably greater than or equal to 23° C. (e.g., α and/or β-crystalline monoglycerides that remain substantially in a crystalline form, such as, greater than or equal to 50%, 55%, 60%, 65%, 70%, 75%, 80%, 85%, 90%, or 95% crystalline form, at temperatures greater than or equal to 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., or 42° C.). Stated differently, the compositions and methods disclosed herein utilize protein/peptide-based formulations (e.g., formulations containing an HGF protein, such as a recombinant, naturally occurring full-length HGF or dHGF or a recombinant naturally occurring variant thereof, for instance, NK1, dNK1, NK2, dNK2, NK3, dNK3, NK4 or dNK4) and one or a mixture of a plurality of monoglycerides (e.g., an α and/or β-crystalline monoglycerides having a carbon chain length of 10, 11, 12, 13, 14, 15, 16, 17, or 18 carbons, preferably, 12 or 14 carbons) that remain in a crystalline form at temperatures less than or equal to 42° C., 41° C., 40° C., 39° C., 38° C., 37° C., 36° C., 35° C., 34° C., 33° C., 32° C., 31° C., 30° C., 29° C., 28° C., 27° C., 26° C., 25° C., 24° C., 23° C., 22° C., 21° C., 20° C., 19° C., 18° C., 17° C., 16° C., or 15° C., preferably at skin temperature. Methods of making and using the afore-mentioned compositions (e.g., in methods of improving, ameliorating, or promoting the healing of a wound or skin cancer or a pre-cancer condition) are also contemplated.

Some embodiments additionally comprise a local anaesthetic drug (e.g., bupivacaine) so as to enhance an antimicrobial effect in combination with the crystalline monoglycerides. That is, in some formulations, it is contemplated that the addition of a local anaesthetic drug (e.g., bupivacaine) to a protein/peptide-containing product that comprises a crystalline monoglyceride (e.g., an α or β-crystalline monoglyceride, which may have a carbon chain length of 10, 11, 12, 13, 14, 15, 16, 17, or 18 carbons, desirably a carbon chain length between 10-16 carbons (e.g., 12, 13, 14, 15, or 16 carbons), preferably a carbon chain length that is 12, 13, or 14 carbons, and most preferably, 12 or 14 carbons) provides an enhanced or synergistic antibacterial effect, as well as, a better stabilized protein/peptide. Preferably, the local anaesthetic agent is included in amounts that are below the amount required for local anaesthesia. Stated differently, in some embodiments, the compositions and methods disclosed herein utilize protein/peptide-based formulations (e.g., formulations containing an HGF protein, such as a recombinant, naturally occurring full-length HGF or dHGF or a recombinant naturally occurring variant thereof, for instance, NK1, dNK1, NK2, dNK2, NK3, dNK3, NK4 or dNK4) and one or a mixture of a plurality of monoglycerides (e.g., an α and/or β-crystalline monoglycerides having a carbon chain length of 10, 11, 12, 13, 14, 15, 16, 17, or 18 carbons, preferably 12 or 14 carbons) that remain in a crystalline form at temperatures less than or equal to 42° C., 41° C., 40° C., 39° C., 38° C., 37° C., 36° C., 35° C., 34° C., 33° C., 32° C., 31° C., 30° C., 29° C., 28° C., 27° C., 26° C., 25° C., 24° C., 23° C., 22° C., 21° C., 20° C., 19° C., 18° C., 17° C., 16° C., or 15° C., preferably at skin temperature, and, additionally include, a therapeutic amount of a local anaesthetic drug (e.g., bupivacaine), which may be present at concentrations that are at or below the amount required for local anaesthesia.

Other embodiments additionally comprise at least one anti-pathogenic compound, in addition to or in lieu of the local anaesthetic drug (e.g., bupivacaine) and the at least one crystalline monoglyceride. In some aspects of the invention, bupivacaine is itself the anti-pathogenic compound and no other antimicrobial agent is provided and in other formulations another anti-pathogenic compound other than bupivacaine is provided. In some embodiments, the aforementioned compositions include an anti-pathogenic compound that is selected from the group consisting of a local anaesthetic of the amide type, a carbamide, an imidazole derivative, a nitroimidazole derivative, a diol with 3-6 carbon atoms, an antibiotic, and an antifungal composition in lieu of or in addition to bupivacaine. That is, aspects of the invention include an anti-pathogenic compound that is selected from the group consisting of a local anaesthetic of the amide type, a carbamide, an imidazole derivative, a nitroimidazole derivative, a diol with 3-6 carbon atoms, an antibiotic, and an antifungal composition in addition to a protein/peptide-containing product that comprises a crystalline monoglyceride (e.g., an α or β-crystalline monoglyceride, which may have a carbon chain length of 10, 11, 12, 13, 14, 15, 16, 17, or 18 carbons, desirably a carbon chain length between 10-16 carbons (e.g., 12, 13, 14, 15, or 16 carbons), preferably a carbon chain length that is 12, 13, or 14 carbons, and most preferably, 12 or 14 carbons), and optionally, may include a local anaesthetic drug, such as bupivacaine. Stated differently, in some embodiments, the compositions and methods disclosed herein utilize protein/peptide-based formulations (e.g., formulations containing an HGF protein, such as a recombinant, naturally occurring full-length HGF or dHGF or a recombinant naturally occurring variant thereof, for instance, NK1, dNK1, NK2, dNK2, NK3, dNK3, NK4 or dNK4) and one or a mixture of a plurality of monoglycerides (e.g., an α and/or β-crystalline monoglycerides having a carbon chain length of 12, 13, 14, 15, 16, 17, or 18 carbons) that remain in a crystalline form at temperatures less than or equal to 42° C., 41° C., 40° C., 39° C., 38° C., 37° C., 36° C., 35° C., 34° C., 33° C., 32° C., 31° C., 30° C., 29° C., 28° C., 27° C., 26° C., 25° C., 24° C., 23° C., 22° C., 21° C., 20° C., 19° C., 18° C., 17° C., 16° C., or 15° C., preferably at skin temperature, and, additionally include, an anti-pathogenic compound that is selected from the group consisting of a local anaesthetic of the amide type, a carbamide, an imidazole derivative, a nitroimidazole derivative, a diol with 3-6 carbon atoms, an antibiotic, and an antifungal composition in lieu of or in addition to a local anaesthetic drug, such as bupivacaine.

Some embodiments additionally comprise one or more viscosity-increasing agents. Preferred viscosity-increasing agents include but are not limited to a cellulose derivative that is selected from the group consisting of hydroxyethylcellulose, methylcellulose, hydroxypropyl cellulose, carboxymethyl cellulose, hydroxypropyl methyl cellulose (E464), and hydroxyethyl methyl cellulose. That is, some embodiments include a viscosity-increasing agent such as a cellulose derivative selected from the group consisting of hydroxyethylcellulose, methylcellulose, hydroxypropyl cellulose, carboxymethyl cellulose, hydroxypropyl methyl cellulose (E464), and hydroxyethyl methyl cellulose in addition to a protein/peptide-containing product that comprises a crystalline monoglyceride (e.g., an α or β-crystalline monoglyceride, which may have a carbon chain length of 10, 11, 12, 13, 14, 15, 16, 17, or 18 carbons, desirably a carbon chain length between 10-16 carbons (e.g., 12, 13, 14, 15, or 16 carbons), preferably a carbon chain length that is 12, 13, or 14 carbons, and most preferably, 12 or 14 carbons), and optionally, may include a local anaesthetic drug, such as bupivacaine and/or an anti-pathogenic compound that is selected from the group consisting of a local anaesthetic of the amide type, a carbamide, an imidazole derivative, a nitroimidazole derivative, a diol with 3-6 carbon atoms, an antibiotic, and an antifungal composition. Stated differently, in some embodiments, the compositions and methods disclosed herein utilize protein/peptide-based formulations (e.g., formulations containing an HGF protein, such as a recombinant, naturally occurring full-length HGF or dHGF or a recombinant naturally occurring variant thereof, for instance, NK1, dNK1, NK2, dNK2, NK3, dNK3, NK4 or dNK4) and one or a mixture of a plurality of monoglycerides (e.g., an α and/or β-crystalline monoglycerides having a carbon chain length of 10, 11, 12, 13, 14, 15, 16, 17, or 18 carbons, preferably 12 or 14 carbons) that remain in a crystalline form at temperatures less than or equal to 42° C., 41° C., 40° C., 39° C., 38° C., 37° C., 36° C., 35° C., 34° C., 33° C., 32° C., 31° C., 30° C., 29° C., 28° C., 27° C., 26° C., 25° C., 24° C., 23° C., 22° C., 21° C., 20° C., 19° C., 18° C., 17° C., 16° C., or 15° C., preferably at skin temperature, and, additionally include, a viscosity-increasing agent such as a cellulose derivative selected from the group consisting of hydroxyethylcellulose, methylcellulose, hydroxypropyl cellulose, carboxymethyl cellulose, hydroxypropyl methyl cellulose (E464), and hydroxyethyl methyl cellulose and, optionally, an anti-pathogenic compound that is selected from the group consisting of a local anaesthetic of the amide type, a carbamide, an imidazole derivative, a nitroimidazole derivative, a diol with 3-6 carbon atoms, an antibiotic, and an antifungal composition and/or, optionally, a local anaesthetic drug, such as bupivacaine.

In many embodiments, the composition is a dry powder and in other embodiments the composition is a reconstituted gel or cream. In some embodiments, the reconstituted gel or cream is obtained by mixing a dry powder comprising at least one crystalline monoglyceride (e.g., an α and/or β-crystalline monoglycerides having a carbon chain length of 10, 11, 12, 13, 14, 15, 16, 17, or 18 carbons, desirably a carbon chain length between 10-16 carbons (e.g., 12, 13, 14, 15, or 16 carbons), preferably a carbon chain length that is 12, 13, or 14 carbons, and most preferably, 12 or 14 carbons that remain in a crystalline form at temperatures less than or equal to 42° C., 41° C., 40° C., 39° C., 38° C., 37° C., 36° C., 35° C., 34° C., 33° C., 32° C., 31° C., 30° C., 29° C., 28° C., 27° C., 26° C., 25° C., 24° C., 23° C., 22° C., 21° C., 20° C., 19° C., 18° C., 17° C., 16° C., or 15° C., preferably at skin temperature) and a viscosity-increasing agent with a liquid comprising a protein/peptide, preferably an HGF protein, such as a recombinant, naturally occurring full-length HGF or dHGF (a five amino acid truncated form of HGF) or a recombinant naturally occurring variant thereof (e.g., NK1, dNK1, NK2, dNK2, NK3, dNK3, NK4 or dNK4).

In other embodiments, the reconstitution is performed inside a multi-compartment device, wherein a dry part of the composition is brought in contact with a fluid part of the composition through a packaging device configured to allow contact between the content in the different compartments when desired (e.g., upon squeezing, inversion, or breaking of a seal) while inhibiting contact with the surroundings (e.g., while maintaining a closed/sterile system). One contemplated device is a sachet with dual inner compartments that are breakable upon pressure allowing the components of the compartments to mix and to initiate the reconstitution reaction.

Aspects of the invention also include methods of making and using the aforementioned compositions. By one approach the compositions described herein are made by providing a solution that comprises a protein or a peptide (e.g., an HGF protein, such as full-length HGF or dHGF (a five amino acid truncated form of HGF) or a variant thereof (e.g., NK1, dNK1, NK2, dNK2, NK3, dNK3, NK4 or dNK4) and at least one monoglyceride that remains in crystalline form at and/or below body temperature (e.g., an α and/or β-crystalline monoglycerides having a carbon chain length of 10, 11, 12, 13, 14, 15, 16, 17, or 18 carbons, preferably 12 or 14 carbons) that remain in a crystalline form at temperatures less than or equal to 42° C., 41° C., 40° C., 39° C., 38° C., 37° C., 36° C., 35° C., 34° C., 33° C., 32° C., 31° C., 30° C., 29° C., 28° C., 27° C., 26° C., 25° C., 24° C., 23° C., 22° C., 21° C., 20° C., 19° C., 18° C., 17° C., 16° C., or 15° C., preferably at skin temperature and drying said solution to form dry granules, wherein the drying process preserves the crystalline structure of the monoglycerides. Many available drying methods can be used to make one or more of the formulations described herein. Preferably, the temperature during drying does not exceed the melting point of the monoglyceride crystals. In some approaches, the solution used in the method further comprises a viscosity-increasing agent such as a cellulose derivative selected from the group consisting of hydroxyethylcellulose, methylcellulose, hydroxypropyl cellulose, carboxymethyl cellulose, hydroxypropyl methyl cellulose (E464), and hydroxyethyl methyl cellulose. Some of the embodied methods also employ a solution that comprises at least one anti-pathogenic compound, either alone or in combination with said at least one crystalline monoglyceride. In preferred methods, the anti-pathogenic compound is bupivacaine; however, the anti-pathogenic compound can be selected from the group consisting of a local anaesthetic of the amide type, a carbamide, an imidazole derivative, a nitroimidazole derivative, and a diol with 3-6 carbon atoms.

By another synthetic approach, a solution that comprises at least one monoglyceride that remains in crystalline form at and/or below body temperature (e.g., an α or β-crystalline monoglyceride, which may have a carbon chain length of 10, 11, 12, 13, 14, 15, 16, 17, or 18 carbons, desirably a carbon chain length between 10-16 carbons (e.g., 12, 13, 14, 15, or 16 carbons), preferably a carbon chain length that is 12, 13, or 14 carbons, and most preferably, 12 or 14 carbons) is combined with a viscosity-increasing agent (e.g., hydroxyethylcellulose, methylcellulose, hydroxypropyl cellulose, carboxymethyl cellulose, hydroxypropyl methyl cellulose (E464), or hydroxyethyl methyl cellulose) and this mixture is dried to form dry granules in a manner that preserves the β-crystalline structure of the monoglycerides. Next, a liquid that comprises a protein or a peptide (e.g., an HGF protein, such as full-length HGF or dHGF (a five amino acid truncated form of HGF) or a variant thereof (e.g., NK1, dNK1, NK2, dNK2, NK3, dNK3, NK4 or dNK4) is provided and said liquid that comprises a protein or a peptide is combined with said dry granules comprising said at least one monoglyceride and a viscosity-increasing agent under conditions that retain the crystalline structure of the at least one monoglyceride. By some approaches, this method uses a viscosity-increasing agent that is a cellulose derivative selected from the group consisting of hydroxyethylcellulose, methylcellulose, hydroxypropyl cellulose, carboxymethyl cellulose, hydroxypropyl methyl cellulose (E464), and hydroxyethyl methyl cellulose. Optionally, the solution used in these methods further comprises at least one anti-pathogenic compound, either alone or in combination with said at least one monoglyceride. Preferably, the anti-pathogenic compound is bupivacaine; however, the anti-pathogenic compound can be selected from the group consisting of a local anaesthetic of the amide type, a carbamide, an imidazole derivative, a nitroimidazole derivative, and a diol with 3-6 carbon atoms.

The compositions described herein can be used in methods to improve or ameliorate a skin condition or to restore the youthful appearance of a subject. By one approach, a subject in need of an agent that improves or ameliorates a skin condition or that restores a youthful appearance of said subject is identified and any one or more of the aforementioned compositions is provided to the identified subject. Subjects in need of an agent that improves or ameliorates a skin condition or that restores a youthful appearance of said subject can be identified by clinical evaluation or diagnostic tests or observation, as is routinely performed by those in the field. In some embodiments the compositions described herein are used to treat, prevent, improve or ameliorate a skin condition such as, chronic diabetic skin ulcer, a laceration, a wound, bedsores, decubitus ulcer, pressure gangrene or a cosmetic blemish. In more embodiments, the compositions described herein are used to improve the youthful appearance of a subject or to ameliorate wrinkles of a subject. Optionally, the improvement or amelioration of the skin condition or restoration of youthful appearance can be measured using conventional diagnostic or clinical evaluation or observation of an improvement or amelioration of the skin condition or youthful appearance after application of one or more of the embodied compositions.

Accordingly, in some aspects of the invention, uses of a mixture comprising a protein or peptide (e.g., HGF protein, such as full-length HGF or dHGF (a five amino acid truncated form of HGF) or a variant thereof (e.g., NK1, dNK1, NK2, dNK2, NK3, dNK3, NK4 or dNK4) and a crystalline monoglyceride (e.g., an α or β-crystalline monoglyceride, which may have a carbon chain length of 10, 11, 12, 13, 14, 15, 16, 17, or 18 carbons, desirably a carbon chain length between 10-16 carbons (e.g., 12, 13, 14, 15, or 16 carbons), preferably a carbon chain length that is 12, 13, or 14 carbons, and most preferably, 12 or 14 carbons); and, optionally, a viscosity-increasing agent (e.g., hydroxyethylcellulose, methylcellulose, hydroxypropyl cellulose, carboxymethyl cellulose, hydroxypropyl methyl cellulose (E464), or hydroxyethyl methyl cellulose), and, optionally, a local anaesthetic of the amide type, a carbamide, an imidazole derivative, a nitroimidazole derivative, or a diol with 3-6 carbon atoms, such as bupivacaine to prepare a medicament for the improvement, amelioration, or treatment of a condition of the skin or for restoring a youthful appearance (e.g., ulcers, diabetic ulcers, lacerations, punctures, abrasions, cosmetic abrasions, bedsores, decubitus ulcer, pressure gangrene, burns, post-surgical traumas, cosmetic reconstructions, psoriasis, hair growth, wrinkle reduction, skin tightness, skin neoplasia, basal cell carcinoma, squamous cell carcinoma, melanoma or pre-cancerous skin conditions, such as actinic keratosis) is contemplated.

Accordingly, some embodiments described herein include a stabilized protein composition comprising a protein or a peptide that has a biological activity; and at least one monoglyceride that has a melting temperature that is greater than or equal to 20° C. In some embodiments, the protein is a recombinant form of a naturally occurring hepatocyte growth factor (HGF) and in some embodiments, the recombinant form of a naturally occurring HGF is a five amino acid truncated form of HGF (dHGF) or a naturally occurring HGF that is selected from the group consisting of NK1, dNK1, NK2, dNK2, NK3, dNK3, NK4 or dNK4. In some of the compositions above, the monoglyceride has a melting temperature that is greater than or equal to 25° C., greater than or equal to 30° C., or greater than or equal to 35° C. In some of the embodiments above, the composition further comprises an antipathogenic agent in addition to said at least one monoglyceride. In some embodiments, said anti-pathogenic agent is selected from the group consisting of a local anaesthetic of the amide type, a carbamide, an imidazole derivative, a nitroimidazole derivative, and a diol with 3-6 carbon atoms and in some embodiments the antipathogenic agent is bupivacaine. The compositions described above can further comprise a viscosity-increasing agent such as a cellulose derivative, selected from the group consisting of hydroxyethylcellulose, methylcellulose, hydroxypropyl cellulose, carboxymethyl cellulose, hydroxypropyl methyl cellulose (E464), and hydroxyethyl methyl cellulose and in some of the embodiments above the at least one monoglyceride has a carbon chain length of 10, 11, 12, 13, 14, 15, or 16 carbons, preferably 12 or 14 carbons.

More embodiments described herein concern a stabilized protein composition comprising a dHGF protein formulated with a β-crystalline monoglyceride having a carbon length of 10, 11, 12, 13, 14, 15, or 16 carbons, preferably 12 or 14 carbons, wherein the concentration of said dHGF in the stabilized protein composition is less than or equal to 50 ng/ml. In some of these embodiments, the formulation comprises 1-glycerol monolaurate or 1-glycerol monomyristate or both and the formulation can also comprise a viscosity-increasing agent and/or an antimicrobial agent. In some embodiments, the viscosity increasing agent is hydroxyethylcellulose and the antimicrobial agent is bupivacaine. In some of the embodiments described herein the concentration of dHGF in the stabilized protein composition is less than or equal to 10 ng/ml.

More aspects to the invention concern a stabilized protein composition comprising an HGF or HGF variant protein formulated with 1-glycerol monolaurate and 1-glycerol monomyristate. In some embodiments, the composition further comprises a viscosity-increasing agent (e.g., hydroxyethylcellulose) and some of the aforementioned compositions also include an antipathogenic agent (e.g., bupivacaine). In some of these compositions, the HGF or HGF variant protein is selected from the group consisting of NK1, dNK1, NK2, dNK2, NK3, dNK3, NK4 or dNK4 or a protein that comprises at least 80%, 85%, 90%, 95%, 96%, 97%, 98%, or 99% (or any % within 85%-99%) sequence identity or homology to said HGF or HGF variant protein with the proviso that said identical or homologous protein retains a function attributed to said HGF or HGF variant protein, such as binding to a cMet receptor, epitheliocyte acceleration, cell scattering, induction of cell growth, inhibition epitheliocyte acceleration, inhibition of cell scattering, or inhibition of cell growth.

Still more aspects to the invention concern a stabilized protein composition comprising a dHGF protein formulated with 1-glycerol monolaurate and 1-glycerol monomyristate. In some embodiments, these compositions further comprise a viscosity-increasing agent (e.g., hydroxyethylcellulose) and some of the aforementioned compositions also include an antipathogenic agent (e.g., bupivacaine). Additional embodiments include a stabilized protein composition comprising an HGF, NK1, dNK1, NK2, dNK2, NK3, dNK3, NK4 or dNK4 formulated with 1-glycerol monolaurate and 1-glycerol monomyristate and these compositions can, optionally include a viscosity-increasing agent (e.g., hydroxyethylcellulose) and some of the aforementioned compositions also include an antipathogenic agent (e.g., bupivacaine).

Aspects of the invention also include methods of making the compositions described above, wherein said methods are practiced by providing a solution that comprises a dHGF protein and at least one monoglyceride that has a melting temperature above 30° C.; and drying said solution to form dry granules, wherein the drying process preserves the β-crystalline structure of the monoglycerides. In some of these methods, the solution further comprises a viscosity-increasing agent. In some of these methods, the viscosity-increasing agent is a cellulose derivative, selected from the group consisting of hydroxyethylcellulose, methylcellulose, hydroxypropyl cellulose, carboxymethyl cellulose, hydroxypropyl methyl cellulose (E464), and hydroxyethyl methyl cellulose and in some of these methods the at least one monoglyceride has a carbon chain length of 10, 11, 12, 13, 14, 15, or 16 carbons, preferably 12 or 14 carbons. In some of these methods, the solution further comprises at least one anti-pathogenic compound, either alone or in combination with said at least one monoglyceride and in some of these methods, the anti-pathogenic compound is bupivacaine. In some of these methods, the anti-pathogenic compound is selected from the group consisting of a local anaesthetic of the amide type, a carbamide, an imidazole derivative, a nitroimidazole derivative, and a diol with 3-6 carbon atoms.

Aspects of the invention also include methods of making the aforementioned compositions comprising providing a solution that comprises at least one crystalline monoglyceride that has a melting temperature above 30° C. and a viscosity-increasing agent; freeze spray-drying said solution to form dry granules, wherein the freeze drying process preserves the β-crystalline structure of the monoglycerides; providing a liquid that comprises a recombinant form of a naturally occurring HGF protein; and combining said liquid that comprises said HGF protein with said dry granules comprising said at least one monoglyceride and a viscosity-increasing agent under conditions that retain the crystalline structure of the at least one monoglyceride. In some of these methods the recombinant form of a naturally occurring HGF protein is dHGF and in some of these methods, the viscosity-increasing agent is a cellulose derivative, selected from the group consisting of hydroxyethylcellulose, methylcellulose, hydroxypropyl cellulose, carboxymethyl cellulose, hydroxypropyl methyl cellulose (E464), and hydroxyethyl methyl cellulose. In some of these methods, the at least one monoglyceride has a carbon chain length of 10, 11, 12, 13, 14, 15, or 16 carbons, preferably 12 or 14 carbons and in some of these methods, the solution further comprises at least one anti-pathogenic compound, either alone or in combination with said at least one monoglyceride. In some of these methods, the anti-pathogenic compound is bupivacaine and in some of these methods the anti-pathogenic compound is selected from the group consisting of a local anaesthetic of the amide type, a carbamide, an imidazole derivative, a nitroimidazole derivative, and a diol with 3-6 carbon atoms.

Aspects of the invention also include methods of using the aforementioned compositions to improve or ameliorate a skin condition of a subject comprising identifying a subject in need of an agent that improves or ameliorates a skin condition; and providing a composition described herein to said subject. In some of these methods, the skin condition is a chronic diabetic skin ulcer, a laceration, a wound, a cosmetic blemish, a skin neoplasia, or a basal cell carcinoma. Optionally, the methods above can further comprise measuring the improvement or amelioration of the skin condition.

### BRIEF DESCRIPTION OF THE DRAWINGS

FIG. 1 shows melting curves of cream ÅLL07001 and a typical curve from one of the dry powders (ÅLL07005C) and two of the reconstituted powders (ÅLL07005A and ÅLL07005F).

### DETAILED DESCRIPTION OF THE PREFERRED EMBODIMENTS

Many embodiments described herein concern topical compositions that contain a stabilized delivered agent (e.g., a protein or fragment thereof and/or a nucleic acid, such as, DNA or RNA). In a preferred embodiment, the composition comprises at least one crystalline monoglyceride, such as a monoglyceride that retains a crystalline structure at or below 42° C., 41° C., 40° C., 39° C., 38° C., 37° C., 36° C., 35° C., 34° C., 33° C., 32° C., 31° C., 30° C., 29° C., 28° C., 27° C., 26° C., 25° C., 24° C., 23° C., 22° C., 21° C., 20° C., 19° C., 18° C., 17° C., 16° C., or 15° C., preferably at or below skin temperature. In a more preferred embodiment, the monoglycerides are in a β-crystalline state. The “crystalline state” is a structure that is repeated in all three dimensions although the repeated function does not have to be the same in all three directions. A β-crystalline monoglyceride contains solid lamellar structures of solid monoglycerides, the carbon chains are not melted but solid.

In another preferred embodiment, the composition further comprises at least one anti-pathogenic compound (e.g., an antimicrobial compound, an antifungal agent, a bacteriocidal or bacteriostatic agent, or an antibiotic), either alone or in combination with the crystalline monoglyceride, and, optionally, an excipient. In a particularly preferred embodiment, the composition further comprises a suspending or viscosity-increasing agent (e.g., a cellulose derivative, such as hydroxy propyl cellulose, hydroxy methyl cellulose, hydroxyethylcellulose (e.g., Natrosol®), methyl cellulose, carboxymethyl cellulose, or hydroxypropyl methyl cellulose).

In some embodiments, the topical composition is manufactured as a dry powder having a liquid-absorbing capacity, which upon exposure to the liquid (e.g., water, oil, or an emulsion) will generate a semisolid formulation. This regenerated semisolid product can be applied topically so as to induce epidermal, dermal, transdermal or intradermal delivery of a delivered agent (e.g., a protein or fragment thereof, a nucleic acid (e.g., DNA or RNA) or combination thereof). In some embodiments, the active ingredient is present in a liquid form (e.g., in a suitable buffer) and the aqueous solution containing the active ingredient is added to a dry powder comprising the gelling agent or viscosity-increasing agent so as to prepare the final formulation of the stabilized product. The mixing of an aqueous solution comprising the active ingredient and the dry powder comprising the gelling agent can be performed slightly before providing the stabilized composition to the subject that has been identified as one in need of the active ingredient.

These formulations of stabilized delivered agents can be used to provide benefit to the health and welfare of a subject (e.g., an animal, domestic animal, reptile, bird, or mammal, such as, a dog, cat, horse, pig, or human). Some formulations are useful to treat, ameliorate, or improve a skin condition of a subject (e.g., promote or accelerate the healing of an ulcer, wound or skin neoplasia). Some embodiments, for example, are formulated to treat, improve, or ameliorate cosmetic conditions or to better the health and welfare of the animal. Some embodiments can be used to treat, improve, ameliorate, ulcers, diabetic ulcers, bedsores, decubitus ulcer, pressure gangrene, lacerations, punctures, abrasions, cosmetic abrasions, burns, post-surgical traumas, skin neoplasias, basal cell carcinomas, squamous cell carcinomas, melanomas, actinic keratosis, cosmetic reconstructions, psoriasis, hair growth, wrinkle reduction, skin tightness, and/or a youthful appearance in the animal, preferably a mammal, such as a human.

Several embodiments concern topical compositions that comprise, consist, or consist essentially of an active ingredient or delivered agent, which is a protein, a nucleic acid encoding a protein or a fragment of a protein or nucleic acid encoding said protein fragment. Exemplary proteins, which can be used as delivered agents in a formulation or product described herein include, but are not limited to, a growth hormone, including human growth hormone (hGH), hepatocyte growth factor or scatter factor (HGF), and des-N-methionyl human growth hormone; parathyroid hormone; thyroid stimulating hormone; thyroxine; lipoproteins; α1-antitrypsin; insulin α-chain; insulin β-chain; proinsulin; clotting factors such as factor VIIIC, factor IX, tissue factor, and von Willebrands factor; anti-clotting factors such as Protein C; atrial natrietic peptide; lung surfactant; a plasminogen activator, such as urokinase or human urine or tissue-type plasminogen activator (t-PA); bombesin; thrombin; hemopoietic growth factor; tumor necrosis factor (TNF) α and β; enkephalinase; a serum albumin such as human serum albumin; mullerian-inhibiting substance; relaxin A-chain; relaxin B-chain; prorelaxin; DNase; inhibin; activin; vascular endothelial growth factor; receptors for hormones or growth factors; integrin; thrombopoietin; protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor such as NGF-β.; platelet-derived growth factor (PDGF); fibroblast growth factor such as aFGF and bFGF; epidermal growth factor (EGF); transforming growth factor (TGF) such as TGF-α and TGF-β, including TGF-β1, TGF-β2, TGF-β3, TGF-β4, or TGF-γ; insulin-like growth factor-I and -II (IGF-I and IGF-II); insulin-like growth factor binding proteins; CD proteins such as CD-3, CD-4, CD-8, and CD-19; erythropoietin; osteoinductive factors; a bone morphogenetic protein (BMP); somatotropins; an interferon (IFN) such as IFN-α, IFN-β, and IFN-γ; colony stimulating factors (CSFs), e.g., M-CSF, GM-CSF, and G-CSF; interleukins (ILs), e.g., IL-1 to IL-10; superoxide dismutase; T-cell receptors; surface membrane proteins; decay accelerating factor; homing receptors; addressins; regulatory proteins; or antibodies.

Particularly preferred active ingredients for incorporation into on or more of the compositions described herein include a recombinantly produced or isolated naturally occurring HGF protein, such as full-length HGF or dHGF (a naturally occurring five amino acid truncated form of HGF) or naturally occurring variants thereof (e.g., NK1, dNK1, NK2, dNK2, NK3, dNK3, NK4 or dNK4) and a crystalline monoglyceride, (e.g., an α and/or β-crystalline monoglycerides having a carbon chain length of 10, 11, 12, 13, 14, 15, 16, 17, or 18 carbons, preferably 12 or 14 carbons, which remain in a crystalline form at temperatures less than or equal to 42° C., 41° C., 40° C., 39° C., 38° C., 37° C., 36° C., 35° C., 34° C., 33° C., 32° C., 31° C., 30° C., 29° C., 28° C., 27° C., 26° C., 25° C., 24° C., 23° C., 22° C., 21° C., 20° C., 19° C., 18° C., 17° C., 16° C., or 15° C., preferably at skin temperature). Aspects of the invention also include formulations that comprise a delivered agent comprising, consisting essentially of, or consisting of a nucleic acid (e.g., DNA, RNA, including inhibitory RNAs, such as RNAi)), which encode and/or interfere with any of the above-listed polypeptides or a mutant thereof. More embodiments include fragments of the above-listed polypeptides and nucleic acids (e.g., DNA, RNA, including inhibitory RNAs, such as RNAi)) encoding and/or interfering with said fragments.

The delivered agents or active ingredients used in some embodiments, for example, include a protein or a nucleic acid encoding a protein having a therapeutic, ameliorative, or improving effect when applied topically to a tissue, such as skin, for example, HGF (hepatocyte growth factor or scatter factor), hGH, TGF-α, TGF-β, platelet-derived growth factor (PDGF), epidermal growth factor (EGF), fibroblast growth factor (FGF), insulin-like growth factor-1 and 2 (IGF-1 and/or IGF-2), t-PA, factor VIII, relaxin, insulin, IFN-γ and/or TGF-γ. Some embodiments may include fragments or mutants of the aforementioned proteins or nucleic acids encoding said fragments or mutants.

In some embodiments, a nucleic acid encoding one or more of the proteins or fragments thereof is included in the formulation (e.g., in addition to the protein or protein fragment or in lieu of said protein or protein fragment). In some embodiments, for example, a nucleic acid encoding HGF, a fragment of HGF, or a mutant version of HGF or mutant version of an HGF fragment (e.g., a full-length HGF or dHGF (a five amino acid truncated form of HGF) or a variant thereof (e.g., NK1, dNK1, NK2, dNK2, NK3, dNK3, NK4 or dNK4)) and a β crystalline monoglyceride (e.g., an α or β-crystalline monoglyceride, which may have a carbon chain length of 10, 11, 12, 13, 14, 15, 16, 17, or 18 carbons, desirably a carbon chain length between 10-16 carbons (e.g., 12, 13, 14, 15, or 16 carbons), preferably a carbon chain length that is 12, 13, or 14 carbons, and most preferably, 12 or 14 carbons), is formulated in a topical product according to the teachings described herein and said formulation may also contain an HGF protein, a fragment of an HGF protein, or a mutant HGF protein or fragment thereof (e.g., a full-length HGF or dHGF (a five amino acid truncated form of HGF) or a variant thereof (e.g., NK1, dNK1, NK2, dNK2, NK3, dNK3, NK4 or dNK4)). That is, in some embodiments, a DNA encoding a protein, e.g., an HGF protein or mutant HGF protein or fragment thereof, may be formulated in a composition described herein with or without an HGF protein or mutant HGF protein or fragment thereof. These nucleic acids can be incorporated into an expression plasmid suitable for the particular subject (e.g., plasmids that are particularly suited to direct expression in a human, cat, dog, horse, pig, or chicken). Further, the DNA delivered agent can be codon-optimized for the particular subject (e.g., human, cat, dog, horse, pig, or chicken) so as to provide improved production of the peptide in the subject. For example, a codon-optimized HGF or mutant HGF DNA (e.g., a full-length HGF or dHGF (a five amino acid truncated form of HGF) or a variant thereof (e.g., NK1, dNK1, NK2, dNK2, NK3, dNK3, NK4 or dNK4) can be formulated in a topical gel given the teachings herein and said codon-optimized HGF or mutant HGF DNA (e.g., a nucleic acid encoding a full-length HGF or dHGF (a five amino acid truncated form of HGF) or a variant thereof (e.g., NK1, dNK1, NK2, dNK2, NK3, dNK3, NK4 or dNK4)) can be optimized for expression in dogs, cats, horses, pigs, or humans.

The proteins/peptides that can be used in the formulations described herein include all natural and synthetic proteins/peptides, whether obtained from natural sources, chemically synthesized, or produced by techniques of recombinant technology. In some embodiments, the proteins/peptides may be glycoproteins, phosphoproteins, iodoproteins, sulphoproteins, methylated proteins, unmodified proteins/peptides or proteins/peptides containing other modifications. Although the compositions described herein can be formulated to contain a wide-range of protein concentrations or can be formulated to deliver a wide-range of protein concentrations, depending on the amount of a particular protein/peptide suitable for therapeutic efficacy, it is preferred that composition is formulated such that the concentration of the protein/peptide contained in the product or delivered by the product is less than or equal to 10 mg/ml, 5 mg/ml, 2 mg/ml, 1 mg/ml, 0.5 mg/ml, 0.2 mg/ml, 0.1 mg/ml, 50 μg/ml, 25 μg/ml, 10 μg/ml, 5 μg/ml, 2 μg/ml, 1 μg/ml, 0.5 μg/ml, 0.2 μg/ml, 0.1 μg/ml, 50 ng/ml, 25 ng/ml, 10 ng/ml, 5 ng/ml, 2 ng/ml, or 1 ng/ml. Stated differently, the compositions or methods described herein can comprise or utilize a concentration of protein or peptide or deliver a concentration protein or peptide that is sufficient to achieve a therapeutic purpose, which can be less than, between, or equal to any number in the ranges of 9-10 mg/ml, 8-9 mg/ml, 7-8 mg/ml, 6-7 mg/ml, 5-6 mg/ml, 4-5 mg/ml, 3-4 mg/ml, 2-3 mg/ml, 1-2 mg/ml, 0.5-1 mg/ml, 0.25-0.5 mg/ml, 0.1-0.25 mg/ml, 0.05-0.1 mg/ml, 0.02-0.05 mg/ml, 10-20 μg/ml, 9-10 μg/ml, 8-9 μg/ml, 7-8 μg/ml, 6-7 μg/ml, 4-5 μg/ml, 3-4 μg/ml, 2-3 μg/ml, 0.5-1 μg/ml, 0.3-0.5 μg/ml, 0.1-0.3 μg/ml, 0.05-0.1 μg/ml, 0.02-0.05 μg/ml, 10-20 ng/ml, 9-10 ng/ml, 8-9 ng/ml, 7-8 ng/ml, 6-1 ng/ml, 5-6 ng/ml, 4-5 ng/ml, 3-4 ng/ml, 2-3 ng/ml, of 1-2 ng/ml depending on the protein/peptide or intended therapy.

With respect to many of the topical compositions for improving, ameliorating, or treating skin conditions such as wounds, ulcers, and skin neoplasias, it is preferred that the amount of active ingredient in the formulation (e.g., a full-length HGF or dHGF (a five amino acid truncated form of HGF) or a variant thereof (e.g., NK1, dNK1, NK2, dNK2, NK3, dNK3, NK4 or dNK4)) is present in a concentration contained in the product or delivered by the product that is any number less than or equal to 5 μg/ml, 4 μg/ml, 3 μg/ml, 2 μg/ml, 1 μg/ml, 0.5 μg/ml, 0.2 μg/ml, 100 ng/ml, 95 ng/ml, 90 ng/ml, 85 ng/ml, 80 ng/ml, 75 ng/ml, 70 ng/ml, 65 ng/ml, 60 ng/ml, 55 ng/ml, 50 ng/ml, 45 ng/ml, 40 ng/ml, 35 ng/ml, 30 ng/ml, 25 ng/ml, 20 ng/ml, 20 ng/ml, 19 ng/ml, 18 ng/ml, 17 ng/ml, 16 ng/ml, 15 ng/ml, 14 ng/ml, 13 ng/ml, 12 ng/ml, 11 ng/ml, 10 ng/ml, 9 ng/ml, 8 ng/ml, 1 ng/ml, 6 ng/ml, 5 ng/ml, 4 ng/ml, 3 ng/ml, 2 ng/ml, or 1 ng/ml.

In a preferred embodiment, the proteins/peptides are formulated for topical delivery as part of a formulation containing a gel forming compound. Any suspending or viscosity-increasing agent can be used in the formulation, including, but not limited to, acacia, agar, alginic acid, aluminum monostearate, bentonite, purified bentonite, magma bentonite, carbomer 934p, carboxymethyl cellulose calcium, carboxymethyl cellulose sodium, carboxymethylcellulose sodium 12, carrageenan, microcrystalline and carboxymethyl cellulose sodium cellulose, dextrin, gelatin, guar gum, hydroxyethylcellulose (e.g., Natrosol®), hydroxypropyl cellulose, hydroxypropyl methylcellulose, magnesium aluminum silicate, methylcellulose, pectin, polyethylene oxide, polyvinyl alcohol, povidone, propylene glycol alginate, silicon dioxide, colloidal silicon dioxide, sodium alginate, tragacanth, and xanthan gum. In a preferred embodiment, cellulose derivatives, including, but not limited to, hydroxyethylcellulose (e.g., Natrosol®), hydroxypropyl cellulose, carboxymethyl cellulose, hydroxypropyl methyl cellulose (E464), and hydroxyethyl methyl cellulose are used as the gel forming compound. The gel forming compound is preferably in an amount that provides a semisolid product. Examples of gel forming compounds include cellulose derivatives, such as described above.

In a preferred embodiment, the formulations envisioned herein contain lipids that are in the solid crystalline state at temperatures below the skin temperature. Lipids used in preferred embodiments have a lipid chain melting temperature that is conducive to the lipids remaining in a solid state at storage temperatures below 42° C. That is, the crystalline monoglycerides that can be used in the compositions or methods described herein have a melting temperature that is less than or equal to 42° C., 41° C., 40° C., 39° C., 38° C., 37° C., 36° C., 35° C., 34° C., 33° C., 32° C., 31° C., 30° C., 29° C., 28° C., 27° C., 26° C., 25° C., 24° C., 23° C., 22° C., 21° C., 20° C., 19° C., 18° C., 17° C., 16° C., or 15° C. The lipid compounds shall be present as part of the dry product in an amount from about 90% to about 1%, preferably from about 85% to about 5%, more preferably from about 80% to about 10%, even more preferably from about 75% to about 15%, still more preferably from about 70% to about 20%, even more preferably from about 65% to about 25% and most preferably from about 60 to about 40%. Examples of preferred lipids used herein include crystalline monoglycerides of fatty acids. The fatty acids include, but are not limited to, saturated fatty acids having, most preferably 12 or 14 carbons, preferably, 10 to 16 carbon atoms and, desirably 10 to 18 carbon atoms. (That is, at least or equal to or any number between 10, 11, 12, 13, 14, 15, 16, 17, or 18 carbon atoms). Preferred compositions comprise crystalline monoglycerides having a carbon chain length of 10, 11, 12, 13, 14, 15, or 16 carbons, including 1-glycerol monolaurate, 1-glycerol monomyristate, 1-glycerol monopalmitate, and 1-glycerol monostearate. Most preferably, the compositions comprise β-crystalline monoglycerides with 12 or 14 carbons. The crystalline monoglycerides can be present in a homogeneous or heterogeneous state, as are commercially available. Preferred monoglycerides used in the compositions and methods described herein include glycerol monolaurate and/or 1-glycerol monomyristate.

Mixtures of crystalline monoglycerides can also be used in some formulations. Accordingly, some embodiments include mixtures of one or more crystalline monoglycerides (e.g., an α or β-crystalline monoglyceride), which may have a carbon chain length of 10, 11, 12, 13, 14, 15, 16, 17, or 18 carbons, desirably a carbon chain length between 10-16 carbons (e.g., 12, 13, 14, 15, or 16 carbons), preferably a carbon chain length that is 12, 13, or 14 carbons, and most preferably, 12 or 14 carbons). Exemplary ratios include 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or 1:20 or greater. Preferable compositions include a mixture of 1-glycerol monolaurate and 1-glycerol monomyristate in a 1:9 or 9:1 ratio.

Since the product is a semisolid, the delivery rate of the protein/peptide can be regulated. This can be performed by alterations in the amount of gel forming compound and in the amount of lipids. The desired rate of delivery may depend on the protein/peptide used and the composition can be tailored for each protein/peptide. The percentage of crystallization of a or β-crystalline monoglycerides may also be important for particular formulations and can be different depending on commercial source, purity, heterogeneity. Accordingly, some embodiments include an amount of a or β-crystalline monoglycerides, wherein the α or β-crystalline monoglycerides have greater than or equal to 50%, 55%, 60%, 65%, 70%, 75%, 80%, 85%, 90%, or 95% or any number in between these percentages of crystalline monoglyceride molecules. In some aspects used herein, the term crystalline monoglyceride refers to a monoglyceride, wherein a greater percentage of the monoglyceride exists in a crystalline state at a particular temperature than in a non-crystalline state. For example, a crystalline monoglyceride can be an α or β-crystalline monoglyceride that has a greater percentage of molecules in a crystalline state than in a non-crystalline state at less than or equal to 42° C., 41° C., 40° C., 39° C., 38° C., 37° C., 36° C., 35° C., 34° C., 33° C., 32° C., 31° C., 30° C., 29° C., 28° C., 27° C., 26° C., 25° C., 24° C., 23° C., 22° C., 21° C., 20° C., 19° C., 18° C., 17° C., 16° C., or 15° C. Such percentages of crystallized form in any particular α or β-crystalline monoglyceride can be determined by differential scanning calorimetry (DSC) analysis.

Additional embodiments concern methods, wherein one or more of the reconstituted gel or cream formulations described herein are provided to a subject in need thereof (e.g., a subject such as a dog, cat, horse, pig, or human to treat, ameliorate, or otherwise improve the condition of, ulcers, diabetic ulcers, bedsores, decubitus ulcer, pressure gangrene, lacerations, punctures, abrasions, cosmetic abrasions, burns, post-surgical traumas, skin neoplasias, basal cell carcinomas, squamous cell carcinomas, melanomas, cosmetic reconstructions, psoriasis, hair growth, wrinkle reduction, skin tightness, and/or a youthful appearance. In such approaches, said subject is preferably identified as a subject in need of a composition that treats, ameliorates, or otherwise improves the condition of one or more of: ulcers, diabetic ulcers, bedsores, decubitus ulcer, pressure gangrene, lacerations, punctures, abrasions, cosmetic abrasions, burns, post-surgical traumas, skin neoplasias, basal cell carcinomas, squamous cell carcinomas, melanomas, actinic keratosis, cosmetic reconstructions, psoriasis, hair growth, wrinkle reduction, skin tightness, and/or a youthful appearance. The identification can be accomplished by clinical or diagnostic evaluation, as is known in the field, which may include consultation with a physician, surgeon, or other health care provider or performing a diagnostic test or biopsy. Such subjects can optionally or alternatively be identified as a subject in need of an agent that induces proliferation of epitheliocytes and/or granulation at a wound site or an agent that inhibits proliferation or scattering of cancer cells. Again, such identification can be accomplished by clinical or diagnostic evaluation, as is known in the field, which may include consultation with a physician, surgeon, or other health care provider, diagnostic evaluation and/or biopsy.

Once the subject is identified, they are provided or administered one or more of the compositions described herein. Preferably, the methods are practiced by providing a composition that is formulated such that the concentration of the protein/peptide contained in the product or delivered by the product is less than or equal to 10 mg/ml, 5 mg/ml, 2 mg/ml, 1 mg/ml, 0.5 mg/ml, 0.2 mg/ml, 0.1 mg/ml, 50 μg/ml, 25 μg/ml, 10 μg/ml, 5 μg/ml, 2 μg/ml, 1 μg/ml, 0.5 μg/ml, 0.2 μg/ml, 0.1 μg/ml, 50 ng/ml, 25 ng/ml, 10 ng/ml, 5 ng/ml, 2 ng/ml, or 1 ng/ml. Stated differently, once the subject is identified as being a patient in need of the particular therapeutic agent, they are provided or administered one or more of the compositions described herein, which depending on the protein/peptide and therapeutic purpose, can comprise or utilize a concentration of protein or peptide or deliver a concentration protein or peptide that is sufficient to achieve the therapeutic purpose, such as less than, between, or equal to any number in the ranges of 9-10 mg/ml, 8-9 mg/ml, 7-8 mg/ml, 6-7 mg/ml, 5-6 mg/ml, 4-5 mg/ml, 3-4 mg/ml, 2-3 mg/ml, 1-2 mg/ml, 0.5-1 mg/ml, 0.25-0.5 mg/ml, 0.1-0.25 mg/ml, 0.05-0.1 mg/ml, 0.02-0.05 mg/ml, 10-20 μg/ml, 9-10 μg/ml, 8-9 μg/ml, 7-8 μg/ml, 6-7 μg/ml, 5-6 μg/ml, 4-5 μg/ml, 3-4 μg/ml, 2-3 μg/ml, 1-2 μg/ml, 0.5-1 μg/ml, 0.3-0.5 μg/ml, 0.1-0.3 μg/ml, 0.05-0.1 μg/ml, 0.02-0.05 μg/ml, 10-20 ng/ml, 9-10 ng/ml, 8-9 ng/ml, 7-8 ng/ml, 6-1 ng/ml, 5-6 ng/ml, 4-5 ng/ml, 3-4 ng/ml, 2-3 ng/ml, of 1-2 ng/ml depending on the protein/peptide and/or intended therapy.

In some preferred methods, wherein an HGF-based formulation is provided, for example, a composition containing a full-length HGF or dHGF (a five amino acid truncated form of HGF) or a variant thereof (e.g., NK1, dNK1, NK2, dNK2, NK3, dNK3, NK4 or dNK4), the composition that is provided is formulated such that the active ingredient above is present in a concentration contained in the product or delivered by the product that is any number less than or equal to 5 μg/ml, 4 μg/ml, 3 μg/ml, 2 μg/ml, 1 μg/ml, 0.5 μg/ml, 0.2 μg/ml, 100 ng/ml, 95 ng/ml, 90 ng/ml, 85 ng/ml, 80 ng/ml, 75 ng/ml, 70 ng/ml, 65 ng/ml, 60 ng/ml, 55 ng/ml, 50 ng/ml, 45 ng/ml, 40 ng/ml, 35 ng/ml, 30 ng/ml, 25 ng/ml, 20 ng/ml, 20 ng/ml, 19 ng/ml, 18 ng/ml, 17 ng/ml, 16 ng/ml, 15 ng/ml, 14 ng/ml, 13 ng/ml, 12 ng/ml, 11 ng/ml, 10 ng/ml, 9 ng/ml, 8 ng/ml, 1 ng/ml, 6 ng/ml, 5 ng/ml, 4 ng/ml, 3 ng/ml, 2 ng/ml, or 1 ng/ml.

The dosing regimen, frequency of administration, and time of delivery can be determined by the physician or other health care provider taking into account the desired application and the particulars of the patient's or subject's condition. In some embodiments, one or more of the compositions described herein are provided every two days and the patient's improvement is monitored and/or measured accordingly. That is, optionally, the subject's recovery or a marker thereof, such as angiogenesis, granulation, appearance of the treated tissue, oozing of the wound, restoration of healthy tissue, disappearance of a neoplastic lesion or keratosis can be measured or monitored by clinical and diagnostic evaluation, as known in the field.

Many wounds or cosmetic skin conditions are infected or are at risk of infection by a pathogen. As the species of pathogen present in the wound is often of “patient origin,” the presence of these organisms does not always result in systemic infection. However, many pathogens can produce proteases that may disrupt the structure and function of some proteins. In certain embodiments, anti-pathogen compounds (e.g., an antimicrobial compound, an antifungal agent, a bacteriocidal or bacteriostatic agent, or an antibiotic), either alone or in combination with the crystalline monoglycerides and protein/peptide active ingredient (e.g., a full-length HGF or dHGF (a five amino acid truncated form of HGF) or a variant thereof (e.g., NK1, dNK1, NK2, dNK2, NK3, dNK3, NK4 or dNK4)) are added to the formulation to inhibit, reduce or treat the pathogen present in the subject.

The inclusion of anti-pathogen compounds in the formulation containing crystalline monoglycerides (preferably β-crystalline monoglycerides) can inhibit, reduce or treat the pathogen synergistically. That is, in some embodiments, it is contemplated that the anti-pathogen compound inhibits proliferation of the pathogen (e.g., bacteria or fungi) synergistically when said compound is applied with a crystalline monoglyceride (preferably β-crystalline monoglyceride). It is also contemplated that in some embodiments, the formulation containing the antipathogen agent, crystalline monoglyceride (preferably β-crystalline monoglyceride), and protein (e.g., a full-length HGF or dHGF (a five amino acid truncated form of HGF) or a variant thereof (e.g., NK1, dNK1, NK2, dNK2, NK3, dNK3, NK4 or dNK4)) will ameliorate, improve, or treat a condition (e.g., a skin condition, such as a skin ulcer) better than a formulation containing the protein/peptide alone or the protein/peptide in combination with the crystalline monoglyceride (preferably β-crystalline monoglyceride) or in combination with the anti-microbial compound. As discussed herein, the combination of particular crystalline monoglycerides (preferably β-crystalline monoglycerides) with certain specific groups of chemical substances provide a synergistic effect with regard to antimicrobial properties (see also U.S. Pat. No. 5,550,145, herein expressly incorporated by reference in its entirety). Antimicrobially effective amounts of a combination of a crystalline monoglycerides (preferably β-crystalline monoglycerides), including, but are not limited to, lauric acid, myristic acid or a blend of these monoglycerides, and at least one chemical substance selected from the following groups: i) a local anaesthetic of the amide type; ii) carbamide; iii) an antimicrobial, antibacterial, or antifungal substance, an imidazole derivative or a nitroimidazole derivative; and iv) a diol with 3-6 carbon atoms are effective to confer antimicrobial properties.

Particularly preferred local anaesthetics of the amide type that can be used in the formulations or methods described herein include, but are not limited to, lidocaine, prilocalne, mepivacaine, cinchocaine, bupivacaine, procaine, dibucaine, tetracaine, oxybuprocaine, oxethazeine and etidocaine. Bupivacaine is the especially preferred substance amongst said local anaesthetics for use in the formulations and methods described herein.

In addition to or in lieu of the local anaesthetics, carbamide compounds, sulfaguanidine, sulfanilylure, urea derivatives, fusidic acid, cephalosporin P can also be used in one or more of the formulations or methods described herein. Imidazole derivatives including econazole nitrate, miconazole nitrate, bifonazole and clotrimazole can also be used in one or more of the formulations or methods described herein. Antipathogenic substances such as nitroimidazole compounds including timidazole and metronidazole can also be used in one or more of the formulations or methods described herein. Diol compounds with 3-6 carbon atoms include, but are not limited to, propanediols (e.g. 1,2-propanediol(propylene glycol,), 1,3-propanediol, 2,2-dimethyl-1,3 propanediol), butanediols (e.g. 1,2-butanediol, 1,3-butanediol(butylene glycol) 1,4-butanediol, 2,3-butanediol), pentanediols (e.g. 1,2-pentanediol(pentylene glycol), 1,3-pentanediol, 1,4-pentanediol, 1,5-pentanediol, 2,3-pentanediol, 2,4-pentanediol) and hexanediols (e.g. 1,2-hexanediol, 1,3-hexanediol, 1,4-hexanediol, 1,5-hexanediol, 1,6-hexanediol, 2,3-hexanediol, 2,4-hexanediol, and 2,5-hexanediol, hexamethylene diol, 1,2-cyclohexane diol, 1,4-cyclohexane diol) can also be used in one or more of the formulations or methods described herein.

In some embodiments, the manufacturing process is designed to generate a dry powder containing the active ingredient or delivered agent (e.g., a full-length HGF or dHGF (a five amino acid truncated form of HGF) or a variant thereof (e.g., NK1, dNK1, NK2, dNK2, NK3, dNK3, NK4 or dNK4)) having a liquid-absorbing capacity. This dry powder has the ability to generate a semisolid upon exposure to water or a suitable buffer. The manufacturing process involves an initial step of dissolution or dispersion of the components and after that drying, removal of the liquid, in combination with formation of particles suitable for reconstitution. In the manufacturing process it can be important to maintain the lipids at a temperature where the crystal structure of the lipids is unchanged. For example, the temperature during manufacture can be lower than the melting temperature of the lipids and for example not to exceed 42° C.

In other embodiments, a manufacturing process is performed to generate a dry powder containing only the gelling agent (e.g., Natrosol®) and monoglyceride and this dry powder is reconstituted with an aqueous solution (e.g., a suitable buffer) containing the active ingredient or delivered agent (e.g., a full-length HGF or dHGF (a five amino acid truncated form of HGF) or a variant thereof (e.g., NK1, dNK1, NK2, dNK2, NK3, dNK3, NK4 or dNK4)). As above, the manufacturing process involves an initial step of dissolution or dispersion of the crystalline monoglyceride (preferably β-crystalline monoglyceride) and gelling agent and after that drying, removal of the liquid, in combination with formation of particles suitable for reconstitution. Preferably, a temperature of less than 35° C. is maintained so that the crystal structure of the lipids remains unchanged. The reconstitution of the powder containing the gelling agent and the crystalline monoglycerides (preferably β-crystalline monoglycerides) can then be performed prior to providing the product to a subject in need hereof or the reconstituted material can be stored until use, preferably at a temperature below room temperature. Non-limiting examples of aspects of the invention are provided below.

### Example 1

Some embodiments described herein include a recombinant human hepatocyte growth factor (rhHGF) with the following sequence:

```
(SEQ. ID. NO. 1)
    1 mwvtkllpal llqhvllhll llpiaipyae gqrkrrntih efkksakttl ikidpalkik
  61 tkkvntadqc anrctrnkgl pftckafvfd karkqclwfp fnsmssgvkk efghefdlye
121 nkdyirncii gkgrsykgtv sitksgikcq pwssmipheh sflpssyrgk dlgenycmp
181 rgeeggpwcf tsnpevryev cdipqcseve cmtcngesyr glmdhtesgk icqrwdhqtp
241 hrhkflpery pdkgfddnyc rnpdgqprpw cytldphtrw eycaiktcad ntmndtdvpl
301 etteciqgqg egyrgtvnti wngipcqrwd sqyphehdmt penfkckdlr enycrnpdgs
361 espwcfttdp nirvgycsqi pncdmshgqd cyrgngknym gnlsqtrsgl tcsmwdknme
421 dlhrhifwep daskinenyc rnpdddahgp wcytgnplip wdycpisrce gdttptivnl
481 dhpviscakt kqlrvvngip trtnigwmvs lryrnkhicg gslikeswvl tarqcfpsrd
541 lkdyeawlgi hdvhgrgdek ckqvinvsql vygpegsdlv lmklarpavl ddfvstidlp
601 nygctipekt scsvygwgyt glinydgllr vahlyimgne kcsqhhrgkv tlneseicag
661 aekigsgpce gdyggplvce qhkmrmvlgv ivpgrgcaip nrpgifvrva yyakwihkii
721 ltykvpqs.
```

More embodiments described herein include dHGF having the following sequence:

```
(SEQ. ID. NO. 2)
    1 mwvtkllpal llqhyllhll llpiaipyae gqrkrrntih efkksakttl ikidpalkik
  61 tkkvntadqc anrctrnkgl pftckafvfd karkqclwfp fnsmssgvkk efghefdlye
121 nkdyirncii gkgrsykgtv sitksgikcq pwssmipheh syrgkdlqen ycrnprgeeg
181 gpwcftsnpe vryevcdipq csevecmtcn gesyrglmdh tesgkicqrw dhqtphrhkf
241 lperypdkgf ddnycrnpdg qprpwcytld phtrweycai ktcadntmnd tdvplettec
301 iqgqgegyrg tvntiwngip cqrwdsqyph ehdmtpenfk ckdlrenycr npdgsespwc
361 fttdpnirvg ycsqipncdm shgqdcyrgn gknymgnlsq trsgltcsmw dknmedlhrh
421 ifwepdaskl nenycrnpdd dahgpwcytg nplipwdycp isrcegdttp tivnldhpvi
481 scaktkqlry vngiptrtni gwmvslryrn khicggslik eswvltarqc fpsrdlkdye
541 awlgihdvhg rgdekckqvl nvsqlvygpe gsdlvlmkla rpavlddfvs tidlpnygct
601 ipektscsvy gwgytgliny dgllrvahly imgnekcsqh hrgkvtlnes eicagaekig
661 sgpcegdygg plvceqhkmr mvlgvivpgr gcaipnrpgi fvrvayyakw ihkiiltykv
721 pqs.
```

### Example 2

A dry powder containing dHGF having a water-absorbing capacity was manufactured. The manufactured composition, before drying, contained approximately 2.5 μg of dHGF, 37.8 g of the β-crystalline monoglyceride 1-glycerol monolaurate, 12.6 g of the β-crystalline monoglyceride 1-glycerol monomyristate, 48 g of hydroxyethylcellulose (e.g., Natrosol®), and water, which brought the composition to 1200 mL.

A mixture of the lipids in water was created, wherein the monoglycerides, 1-glycerol monolaurate and 1-glycerol monomyristate, were mixed with 200 g of water and heated to 70 to 75° C. After 15 minutes of mixing at 70 to 75° C., the lipid solution was slowly cooled to 20° C. to 30° C. to provide the β-crystalline monoglycerides. The remaining fraction of water was used to dissolve the gel forming compound, hydroxyethylcellulose (e.g., Natrosol®), and to disperse the dHGF. The three mixtures or solutions were mixed and spray-dried to a final water content of less than 5% (e.g., frozen in a container having a bottom layer of liquid nitrogen). The frozen particles of the product were collected and freeze dried to less than 5% of water.

### Example 3

In other embodiments, a dry powder having only the gelling agent and a monoglyceride is manufactured and this dried powder is reconstituted in a solution containing dHGF (e.g., a suitable buffer). The manufactured composition, before drying, will contain approximately 37.8 g of 1-glycerol lmonolaurate, 12.6 g of 1-glycerol monomyristate, 48 g of hydroxyethylcellulose (e.g., Natrosol®), and water to bring the composition to 1200 mL.

A lipid mixture is created, wherein the monoglycerides, 1-glycerol monolaurate and 1-glycerol monomyristate, are mixed with a 200 g of the water and heated to 70 to 75° C. After 15 minutes of mixing at 70 to 75° C., the lipid solution is slowly cooled to 20° C. to 30° C. to provide the O-crystalline monoglycerides. The remaining fraction of water is used to dissolve the hydroxyethylcellulose (e.g., Natrosol®). The two mixtures or solutions (i.e., the monoglyceride mixture and the hydroxyethylcellulose mixture) are then mixed and spray-dried to a final water content of less than 5% (e.g., frozen in a container having a bottom layer of liquid nitrogen. The frozen particles of the product are collected and freeze dried to less than 5% of water. The freeze-dried powder can then be reconstituted in a solution containing dHGF (e.g. a suitable buffer containing approximately 2.5 μg of dHGF). The reconstituted product forms a gel ready for topical application to a wound.

### Example 4

In this experiment, the crystallinity of various monoglyceride preparations was determined by analyzing the energy requirement of the preparations upon heating using differential scanning calorimetry (DSC). The crystallinity of a cream containing β crystalline monoglycerides and water, ÅLL07001 (see Table 1), was compared with powders, ÅLL07005A and ÅLL07005F (see Table 2), which were manufactured as set forth in Example 2, and reconstituted prior to the DSC analysis. The ÅLL07005C powder was not reconstituted and was included as a control.

TABLE 1
Cream composition

| | ÅLL07001 |
|---|---|
| Ingredients | Intended % (w/w) |
| 1-Glycerol monolaurate | 21.0 |
| 1-Glycerol monomyristate | 7.0 |
| Sodium hydroxide, 1 mM | 0.14 |
| Sodium chloride | 0.8 |
| Bupivacaine HCl | 3.5 |
| Water | 67.6 |
| Total | 100 |
| pH | Between 4 and 6 |

TABLE 2
Composition of powders before drying

| Components/Powder | ÅLL07005A | ÅLL07005C | ÅLL07005F |
|---|---|---|---|
| Monoglycerides | 7% | 6% | 2% |
| Sodium alginate | 2% | 2% | 3% |
| Water | To 100% | To 100% | To 100% |

The ÅLL07001 cream was manufactured with buffer. Water was added to a manufacture container and bupivacaine HCl and NaCl were added, and the pH was controlled (about 5). The water phase was heated to 75° C. with mechanical stirring and then the lipids were added. The mixture was kept at 75° C. for 15 minutes and then the temperature was decreased relatively fast to 35° C. while stirring. The formulation was then kept at this temperature for about 15 minutes, while crystallisation took place (until the cream had become shiny and high viscous). The temperature was then decreased to room temperature during slow stirring.

The crystalline monoglycerides in the form of a cream were mixed with the dry alginate particles by stirring. Additional water was added since the mass to be freeze dried should be fluid. The mixtures were then spray frozen into liquid nitrogen, using CO₂ (g) as spray gas. The nitrogen was evaporated at −34° C. for 3 hours. The mixtures were freeze dried for 21 hours in total.

The melting behavior of the products and raw materials was then analyzed with DSC (Perkin Elmer DSC 7) using the following program:

Cooling to −35° C. for 5 minutes

Heating from −35° C. to 80° C. at 10° C./min.

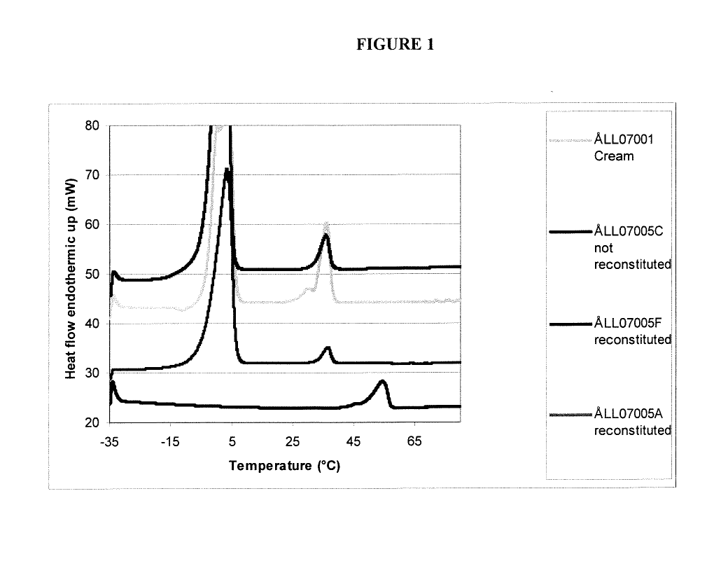

The melting point of the reconstituted powders (ÅLL07005A and ÅLL07005F) and the cream (ÅLL07001), peak in energy requirement, at about 35 C.°, which conforms well to known melting temperatures of β-crystalline monoglycerides in water (see FIG. 1). The melting point of the non-reconstituted powder at over 50° C. is in accordance with the melting temperatures of dry monoglycerides. These data show that the β-crystalline crystals that were formed during manufacture of the cream survived the drying process and were reformed upon exposure to water.

### Example 5

The powder dHGF formulation prepared in accordance with the methods described in Example 2 was diluted five times with diluent buffer and the presence and/or recovery of dHGF was determined by ELISA analysis using a commercially available kit. The results showed that the preparatory method yielded a recovery of greater than 90% of the charged dHGF.

### Example 6

The effect of various monoglycerides on the stability of dHGF formulations was then analyzed. In this experiment, the stability of a composition comprising dHGF and β-crystalline monoglycerides was compared with the stability of a composition comprising dHGF and α-crystalline monoglycerides and a composition comprising dHGF and noncrystalline material.

The following compositions were manufactured:

Formulation 6A*

| | |
|---|---|
| 1-Glycerol monolaurate | 12.6 g |
| 1-Gycerol monomyristate | 38.4 |
| Water | 149 g |

*This composition contains about 80% β-crystals, according to DSC analysis.

Formulation 6B**

| | |
|---|---|
| 1-Glycerol monostearate | 40 g |
| Polyethylene stearate (Myrj 62) | 4 g |
| Water | 156 g |

**This composition comprises about 70% α crystals.

Formulation 6C***

| | |
|---|---|
| 1-Glycerol monooleate | 40 g |
| Water | 160 g |

***This composition is non-crystalline.

The formulations were heated to 75° C. stirred for 15 minutes and slowly, during 20 minutes, cooled to room temperature, 20° C. so as to form monoglyceride crystals. Formulation 6A and 6B were off-white creams while formulation 6 C was translucent and contained supernatant water. The crystalline structure was confirmed by microscopic evaluation.

Approximately 10.0 g of formulations 6A, 6B, and 6C were then diluted 1:3 with a buffer containing dHGF and stored in 10 ml test tubes at 2 to 8° C. and at room temperature, approximately 20° C. Samples were withdrawn and frozen after manufacture and after 7 days of storage. The stability of dHGF was evaluated by Elisa (B-Bridge) and the results are shown in Table 3.

TABLE 3
Stability of dHGF after prolonged storage at approximately 20° C.

| | % recovery 7 days after | % decrease in recovery after |
|---|---|---|
| Formulation | manufacture | manufacture |
| 6A | 87.5 | 12.5 |
| 6B | 78.5 | 21.5 |
| 6C | 60.9 | 39.1 |

These results provided strong evidence that crystalline monoglycerides, in particular β-crystalline monoglycerides, drastically improve the stability and storage of proteins and peptide-containing formulations, especially dHGF-containing formulations, which in turn improves the ability to selectively dose and/or deliver specific amounts of the protein to a targeted site vis a vis topical application. This discovery is especially important for molecules like HGF and its variants, e.g., dHGF, since many of the body's responses to the protein, including epitheliocyte proliferation and scattering are tightly regulated by the localized amount of the protein.

### Example 7

Two different topical HGF formulations were evaluated in a pre-clinical study conducted on Gottingen SPF minipigs that had been given experimentally-induced wounds. Accordingly, each formulation was applied to four female Gottingen SPF minipigs that had received the artificial wounds and the healing of each wound was monitored/measured and evaluated over time.

After the acclimatisation period, the body weight of the animals was approximately 31.1-37.7 kg. On arrival, all animals were vaccinated against Lawsonia intracellularis by oral administration (2.0 ml/animal) of Enterisol® Ileitis vet (Boehringer Ingelheim). A pre-treatment period of 3 weeks (including an acclimatisation period of 5 days) was allowed during which the animals were observed daily in order to reject animals in poor condition. The first dHGF formulation (“standard composition”) was prepared as described in U.S. patent application Ser. No. 10/398,304 to Nayeri and contained 20.9 ng/ml dHGF, albumin, heparin, and diluted buffer solution (see below).

Standard dHGF Composition:
Treatment 1: dHGF 20.9 ng/ml
Treatment 2: dHGF Placebo (control)
The inventive stabilized formulation was prepared as described in Example 2 and contained 10 ng/ml dHGF (see below).
Stabilized dHGF Composition
Treatment 3: dHGF 10 ng/ml
Treatment 4: dHGF Placebo (control)

Prior to application of the dHGF products, the surrounding skin was cleaned with sterile water and if necessary shaved. In addition, possible remains of test item from the previous day's dosing were gently removed by use of sterile gauze and sterile water (if necessary).

With respect to the testing of the standard dHGF compositions, the 20.9 ng/ml standard compositions were applied topically immediately after wounding and daily for 14 days thereafter on circular full-thickness wounds (20 mm in diameter) on the minipigs. After this treatment, adverse effects on the wound healing process in comparison to control treated wounds (placebo) were not observed. Slight improvements in wound healing were observed (see Table 4).

With respect to the stabilized dHGF formulation, the results were dramatic (see Table 4). In all wounds, treated with the dHGF 10 ng/ml formulations, the process of healing was advanced; characterized by the presence of well organized granulation tissue in all wounds (graded massive in all wounds). Re-epithelialization was graded massive in 14/24 wounds in the low-dose, 10 ng/ml, group, which was significantly higher than observed in the placebo groups (3/24 in the control group).

TABLE 4
Wound healing effect in minipig

| | % of wound | % of wound | % of wound |
|---|---|---|---|
| | closure rate | closure rate | closure rate |
| Formulation | after 8 days | after 13 days | after 14 days |
| Treatment 1 | 33.2 | — | 52.2 |
| Standard dHGF 20.9 ng/ml | | | |
| Treatment 2 | 30.7 | — | 49.5 |
| Standard dHGF Placebo | | | |
| Treatment 3 | 36.3 | 59.5 | — |
| Stabilized dHGF 10 ng | | | |
| Treatment 4 | 29.4 | 50.4 | — |
| Stabilized dHGF Placebo | | | |

Accordingly, this experiment demonstrated that the stabilized dHGF formulation was significantly more efficient at reepithelialization than the placebo; whereas the standard HGF formulation was not. Additionally, the results show that the stabilized dHGF formulation had an improved effect on wound closure over placebo and standard formulation.

1. A stabilized protein composition comprising:
a recombinant form of a naturally occurring hepatocyte growth factor (HGF); and
at least one monoglyceride that has a melting temperature that is greater than or equal to 20° C.

2-57. (canceled)

58. The stabilized protein composition of claim 1, wherein said recombinant form of a naturally occurring HGF is selected from the group consisting of a five amino acid truncated form of HGF (dHGF), NK1, dNK1, NK2, dNK2, NK3, dNK3, NK4 and dNK4.

59. The stabilized protein composition of claim 1, wherein said monoglyceride has a melting temperature that is greater than or equal to 25° C., 30° C., or 35° C.

60. The stabilized protein composition of claim 1, wherein said composition further comprises a local anaesthetic of the amide type, bupivacaine, a carbamide, an imidazole derivative, a nitroimidazole derivative, or a diol with 3-6 carbon atoms.

61. The stabilized protein composition of claim 1, further comprising a viscosity-increasing agent selected from the group consisting of hydroxyethylcellulose, methylcellulose, hydroxypropyl cellulose, carboxymethyl cellulose, hydroxypropyl methyl cellulose (E464), and hydroxyethyl methyl cellulose.

62. The stabilized protein composition of claim 1, wherein said at least one monoglyceride has a carbon chain length of 10, 11, 12, 13, 14, 15, or 16 carbons.

63. The stabilized protein composition of claim 1, wherein said at least one monoglyceride comprises 1-glycerol monolaurate or 1-glycerol monomyristate.

64. The stabilized protein composition of claim 1, wherein said composition comprises dHGF, 1-glycerol monolaurate, 1-glycerol monomyristate, hydroxyethylcellulose, and bupivacaine.

65. The stabilized protein composition of claim 64, wherein said the amount of dHGF is less than or equal to 50 ng/ml.

66. The stabilized protein composition of claim 64, wherein said the amount of dHGF is less than or equal to 10 ng/ml.

67. A method of making a stabilized protein composition, comprising:
providing a solution that comprises at least one crystalline monoglyceride that has a melting temperature above 30° C. and a viscosity-increasing agent;
freeze spray-drying said solution to form dry granules, wherein the freeze drying process preserves the β-crystalline structure of the monoglycerides;
providing a liquid that comprises a recombinant form of a naturally occurring HGF protein; and
combining said liquid that comprises said HGF protein with said dry granules comprising said at least one monoglyceride and a viscosity-increasing agent under conditions that retain the crystalline structure of the at least one monoglyceride.

68. The method of claim 67, wherein said recombinant form of a naturally occurring HGF protein is dHGF.

69. The method of claim 67, wherein said viscosity-increasing agent is selected from the group consisting of hydroxyethylcellulose, methylcellulose, hydroxypropyl cellulose, carboxymethyl cellulose, hydroxypropyl methyl cellulose (E464), and hydroxyethyl methyl cellulose.

70. The method of claim 67, wherein said at least one monoglyceride has a carbon chain length of 10, 11, 12, 13, 14, 15, or 16 carbons.

71. The method of claim 67, wherein said solution further comprises a local anaesthetic of the amide type, bupivacaine, a carbamide, an imidazole derivative, a nitroimidazole derivative, or a diol with 3-6 carbon atoms.

72. The method of claim 67, wherein said naturally occurring HGF protein is dHGF and the amount of dHGF is less than or equal to 50 ng/ml.

73. The method of claim 72, wherein said the amount of dHGF is less than or equal to 10 ng/ml.

74. A method of using the composition of claim 58 to improve or ameliorate a skin condition of a subject comprising:
identifying a subject in need of an agent that improves or ameliorates a skin condition; and
providing the composition of claim 58 to said subject.

75. The method of claim 74, wherein said skin condition is selected from the group consisting of a chronic diabetic skin ulcer, a laceration, a wound, a cosmetic blemish, a skin neoplasia, and a basal cell carcinoma.

76. The method of claim 75, further comprising measuring the improvement or amelioration of the skin condition.
